(19) United States
(12) Patent Application Publication
(10) Pub. No.: US 2016/0095639 A1
(43) Pub. Date: Apr. 7, 2016

(54) WASHER ASSEMBLY FOR SPINAL FIXATION SCREW

(71) Applicant: Nexxt Spine, LLC, Noblesville, IN (US)

(72) Inventors: Andrew J. Elsbury, Fortville, IN (US); Eric J. Lintula, Parker, CO (US); Robert D. Thomas, II, Greenfield, IN (US)

(21) Appl. No.: 14/874,411

(22) Filed: Oct. 3, 2015

Related U.S. Application Data
(60) Provisional application No. 62/059,952, filed on Oct. 5, 2014.

(51) Int. Cl. A61B 17/86

(52) U.S. Cl. CPC A61B 17/8685; A61B 17/8695; A61B 17/866

(57) ABSTRACT

A washer for use with a bone screw having a shank and a head defining a maximum diameter, includes a body having a bottom face for engaging the surface of a bone and an opening therethrough sized to receive the shank of a bone screw extending therethrough. The washer includes a cavity in communication with the opening at the bottom face and configured to receive the head of the bone screw when the shank extends through the opening at said bottom face. The washer includes a plurality of flaps distributed around the opening of the cavity that are bendable from a first configuration in which the flaps define an opening sized to receive the head of the bone screw, to a second configuration in which the flaps define an opening smaller than the maximum diameter of the head of the bone screw.

### CROSS-REFERENCE TO RELATED APPLICATION AND PRIORITY CLAIM

This application is a utility filing from and claims priority to provisional application No. 62/059,952, filed on Oct. 5, 2014, the entire disclosure of which is incorporated herein by reference.

### BACKGROUND

The present disclosure relates to orthopaedic fixation systems, and particularly to screw assemblies for spinal fixation.

One form of spinal fixation is a facet fixation procedure in which an inter-spinous joint is stabilized to assist an interbody implant in the fusion of adjacent vertebrae. In this procedure, a bone screw and washer assembly is driven through the inferior articular process or facet joint, and into the pedicle of the adjacent vertebra.

One problem encountered with facet fixation procedures is maintaining the position of the washer as the bone screw is driven through the facet. In particular, the washer may slide down the shaft of the bone screw and into the bone threads, thereby preventing or adversely affecting screw insertion. Prior approaches to address this problem include a locking ring or collar, thereby adding to the cost, complexity, prominence and reliability of the screw/washer assembly.

Another problem is that bone screws can place the facet joint, which is small and weak, under significant stress. Polyaxial screws help disperse the fixation pressure over the entire joint. However, the spherical portion at the base of the screw head contacts the facet joint before the washer, thereby creating a stress riser in the weak joint. There is a need for a washer assembly that avoids these problems.

### SUMMARY

A washer assembly for use with a facet bone screw comprises s a body having a bottom face for engaging the surface of a bone and an opening therethrough sized to receive the shank of a bone screw extending therethrough. The washer includes a cavity in communication with the opening at the bottom face and configured to receive the head of the bone screw when the shank extends through the opening at said bottom face. The washer includes a plurality of flaps distributed around the opening of the cavity that are bendable from a first configuration in which the flaps define an opening sized to receive the head of the bone screw, to a second configuration in which the flaps define an opening smaller than the maximum diameter of the head of the bone screw.

### DESCRIPTION OF THE FIGURES

FIG. 1 is a perspective view of a bone screw and washer for implantation in the facet joint of the spine.

FIG. 2 is a perspective view of the bone screw and washer of FIG. 1 shown in their assembled condition.

FIG. 3 is a side cross-sectional view of the bone screw and washer of FIG. 1 with the washer in a first configuration for assembly.

FIG. 4 is a side cross-sectional view of the bone screw and washer of FIG. 1 with the washer in a second configuration for assembly.

FIG. 5 is a side cross-sectional view of the assembled bone screw and washer of FIG. 2 shown with the washer at an angle relative to the head of the bone screw.

### DETAILED DESCRIPTION

For the purposes of promoting an understanding of the principles of the invention, reference will now be made to the embodiments illustrated in the drawings and described in the following written specification. It is understood that no limitation to the scope of the invention is thereby intended. It is further understood that the present invention includes any alterations and modifications to the illustrated embodiments and includes further applications of the principles of the invention as would normally occur to one skilled in the art to which this invention pertains.

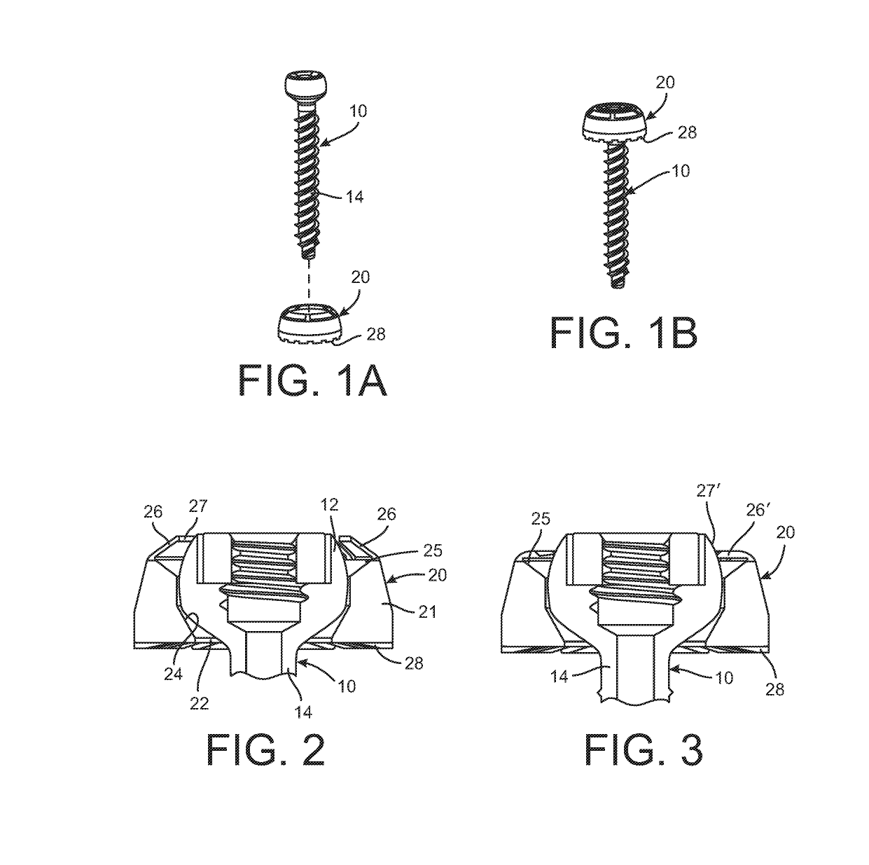

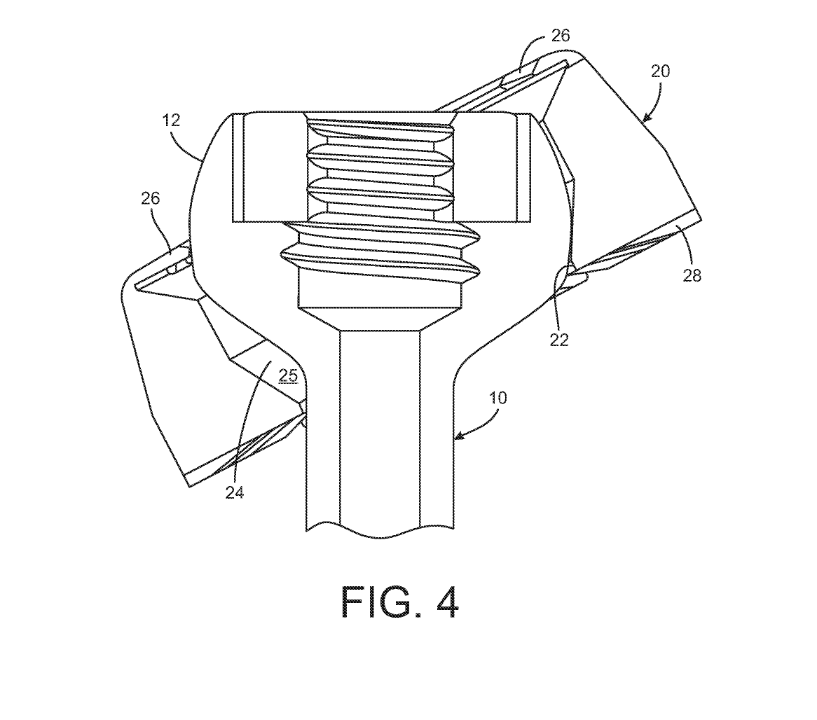

As shown in FIGS. 1-4, a bone screw 10 having a spherical head 12 is disposed within the cavity 24 defined in the body 21 of a washer 20 with the shank 14 of the screw extending through a central opening 22 of the washer. The cavity 24 has an opening 25 at the upper portion of the washer sized to receive the bone screw and is configured for a snug fit around the spherical head 12 of the bone screw with sufficient play to allow the bone screw to move through a range of spherical angles, as depicted in FIG. 4. In the illustrated embodiment, the cavity 24 is generally conical to receive a spherical bottom surface of the bone screw head 12. Other configurations of cavity and bone screw head are contemplated that allow for a snug fit between the screw head and washer.

The upper portion of the washer includes a number of flaps 26 that are initially oriented in a first configuration to define an opening 27 that is large enough in diameter for passage of the head 12 of the bone screw. As shown in FIGS. 1-2, the flaps may be oriented at an inward angle in the first configuration. The inward angle can be about 45° so that the flaps 26 are not very prominent on the washer. The flaps 26 are configured to be bent inward toward the screw head in a second configuration, as shown by the flaps 26′ in FIG. 3. The flaps can be bent to a second configuration in which they are generally parallel with the bottom face 28 of the washer 20. When the flaps are bent inward they define a new smaller diameter 27′ that is smaller than the maximum diameter of the screw head. Thus, when the bone screw is seated within the washer cavity 24, as shown in FIG. 3, the flaps 26′ trap the head of the bone screw within the cavity, thereby holding the washer on the bone screw.

In use, the washer 20 can be installed on a bone screw 10 to be implanted within the facet joint of a patient's spine. The flaps are then bent about the upper portion of the head of the bone screw using an appropriate tool, such as a pliers or similar tool. The head 12 of the bone screw is trapped within the recess 24 by the bent flaps 26′ but the flaps, and particularly the inner opening 27′ of the bent flaps, allows the washer to be moved relative to the bone screw, as depicted in FIG. 4. Consequently, the washer does not interfere with the surgeon's ability to manipulate the bone screw as necessary to align the screw with the facet joint and pedicle or with the surgeon's ability to thread the bone screw into the bone. The head of the bone screw may be provided with a conventional hex recess to receive a driving tool for threading the screw into the bone. Again, the bent flaps 26′ do not interfere with the tool recess or with the driving tool engaged in the recess. The ability of the washer 20 to angulate relative to the bone screw allows the washer to be properly seated on the surface of the bone. The bottom face 28 of the washer may be provided with a surface feature to enhance the engagement with the surface of the facet. The surface feature may include knurling, splines, ridges or similar features that can grip the surface of the bone.

In one embodiment, five flaps 26 are uniformly distributed around the circumference of the washer. The washer is formed of a suitable medical grade metal that permits bending of the flaps, such as stainless steel or titanium. The flaps can be integral with the body of the washer or can be formed as a ring affixed to the upper extent of the washer body.

In a further feature best seen in FIG. 4, the cavity 24 includes a relieved conical segment 25 that supports the head 12 of the bone screw so that no part of the head extends below the bottom face 28 of the washer. This avoids the stress riser that occurs with direct contact of the spherical head and the facet. Instead, the contact is uniform with the flush engagement of the bottom face 28 of the washer with the bone. This contact uniformly distributes the fixation load throughout the entire circumference of the washer. In one embodiment, the recess 24 and relieved conical segment 25 are configured to permit a maximum screw angulation of about 28 degrees.

While the invention has been illustrated and described in detail in the drawings and foregoing description, the same should be considered as illustrative and not restrictive in character. It is understood that only the preferred embodiments have been presented and that all changes, modifications and further applications that come within the spirit of the invention are desired to be protected.

What is claimed is:

1. A washer for use with a bone screw having a shank and a head defining a maximum diameter, the washer comprising:
a body having a bottom face for engaging the surface of a bone and an opening therethrough sized to receive the shank of a bone screw extending therethrough;
a cavity defined in said body in communication with said opening at said bottom face, said cavity having an opening at the upper face of the body opposite said bottom face, said cavity configured to receive the head of the bone screw when the shank extends through the opening at said bottom face; and
a plurality of flaps distributed around said opening of said cavity, said flaps bendable from a first configuration in which the flaps define an opening sized to receive the head of the bone screw therethrough to a second configuration in which the flaps define an opening smaller than the maximum diameter of the head of the bone screw.

2. The washer of claim 1, wherein said cavity includes a conical portion adjacent said bottom face, said conical portion sized so that no part of the head of the bone screw extends below said bottom face of the washer when the head of the bone screw is seated within the recess.

3. The washer of claim 1, wherein said bottom face includes a surface feature for engaging the surface of a bone when the washer is engaged to the bone by the bone screw.

4. The washer of claim 1, wherein said recess is configured to permit relative angulation between the washer and the bone screw when the bone screw is disposed in the cavity and the flaps are in the second configuration.

5. The washer of claim 1, wherein the washer is formed of a biocompatible metal.

6. A bone screw assembly comprising:
a bone screw having a shank and a head defining a maximum diameter; and
a washer including;
a body having a bottom face for engaging the surface of a bone and an opening therethrough sized to receive the shank of a bone screw extending therethrough;
a cavity defined in said body in communication with said opening at said bottom face, said cavity having an opening at the upper face of the body opposite said bottom face, said cavity configured to receive the head of the bone screw when the shank extends through the opening at said bottom face; and
a plurality of flaps distributed around said opening of said cavity, said flaps bendable from a first configuration in which the flaps define an opening sized to receive the head of the bone screw therethrough to a second configuration in which the flaps define an opening smaller than the maximum diameter of the head of the bone screw.

7. The bone screw assembly of claim 6, wherein said cavity includes a conical portion adjacent said bottom face, said conical portion sized so that no part of the head of the bone screw extends below said bottom face of the washer when the head of the bone screw is seated within the recess.

8. The bone screw assembly of claim 6, wherein said bottom face includes a surface feature for engaging the surface of a bone when the washer is engaged to the bone by the bone screw.

9. The bone screw assembly of claim 6, wherein the head of said bone screw is spherical.
